(19) United States
(12) Patent Application Publication
(10) Pub. No.: US 2005/0063980 A1
(43) Pub. Date: Mar. 24, 2005

(54) Gastric raft composition

(76) Inventors: Gillian Eccleston, Glasgow, Central Scotland (GB); Ronald Paterson, Leicestershire (GB)

Correspondence Address: Janis K Fraser;Fish & Richardson, 225 Franklin Street, Bostom, MA, 02110-2804, US

(21) Appl. No.: 10/493,720

(22) PCT Filed: Oct. 29, 2002
(86) PCT No.: PCT/SE02/01957

(30) Foreign Application Priority Data
Oct. 30, 2001 (SE) 0103722-5

(51) Int. Cl. A61K 39/00; A61K 39/38

(52) U.S. Cl. 424/184.1

(57) ABSTRACT

The present invention relates to a gastric raft composition comprising alginic acid or a salt thereof, pectin and a gas producing material capable of producing a non-toxic gas when contacted with aqueous acid. The pectin is preferable a high ester pectin, such as methyl ester pectin and the alginic acid or the salt thereof is preferably of high guluronate content, such as for example sodium or potassium alginate. The gas producing material can be selected from sodium or potassium bicarbonate and the composition can further comprise a pharmaceutically acceptable ingredient, which is an antacid.

The present invention relates to a novel biopolymer gastric raft composition and the use thereof.

### BACKGROUND OF THE INVENTION

A novel pharmaceutical composition comprising alginic acid or a salt thereof, pectin and a gas producing material capable of producing a non-toxic gas when contacted with aqueous acid; where said composition is capable of forming floating gastric rafts on ingestion is provided.

Gastro-oesphagael reflux disease (GORD) occurs when small amounts of gastric fluids and/or bile acids pass into the lower part of the oesophagus and cause oesophageal irritation. Typically, floating rafts are used in the treatment this condition, and are particularly useful for the treatment of GORD in pregnant women and infants due to the rafts having a non-systemic mode of action and generally recognised as safe (GRAS) listed ingredients. On ingestion of a gastric raft composition, the raft forms and acts as a physical barrier on the surface of the gastric contents, preventing reflux of acid and food into the oesophagus. In more severe cases of reflux the raft protects the oesophagael mucosa from further irritation by the low pH gastric fluids.

Gastric raft compositions usually contain biopolymers that react with stomach acid to form gels, which are sufficiently buoyant to float on the gastric contents. Buoyancy is often achieved by the incorporation into the composition of a material capable of producing a non-toxic gas when contacted with aqueous acid. The gas is usually carbon dioxide and typically results from the reaction of the bicarbonate of an alkali or alkaline earth metal with the aqueous acid of the stomach.

Current commercially available gastric raft compositions,.such as the market leading composition Gavison® (Marion Laboratories) are based on an alginate biopolymer. In the Gavison® formulation, sodium alginate (5% w/v) forms a gel when it interacts with calcium ions liberated at low pH from calcium carbonate present in the composition (1.6% w/v). Also known is the floating raft composition of the Boots Company which is based on the biopolymer pectin. This formulation also relies on the interaction of the biopolymer with liberated calcium ions to form a gel. Other floating raft compositions disclosed in the art use xanthan gum as the gel forming ingredient (U.S. Pat. No. 5,360,793).

Whilst a range of gastric raft compositions are known, we can envisage an mre advantageous and improved formulation. The commercial alginate composition, Gavison® liquid has been shown to form rafts over a narrow pH range of hydrochloric acid in vitro (pH 1 to 1.4). At higher pH (greater than pH 1.7) in vitro, no gel forms. It is known that the pH of human gastric juices is highly variable, commonly pH 1.4 to 2.1 for healthy volunteers (Dressman et al., Pharmaceutical Research, Vol 7, No 7 1998). It would therefore be advantageous to formulate a composition capable of forming rafts over a wider range of pH.

Pectin based compositions, like alginate based compositions rely on the presence calcium ions but are also dependent on sugar concentration. Optimum gel strength is highly dependent on there being a high concentration of sugar present, a condition which is not always fulfilled in the gastrointestinal tract. It would therefore be further advantageous to formulate a composition where the strength of the gel formed when exposed to low pH was not dependent on sugar concentration.

In accordance with the present invention there is therefore provided a composition comprising alginic acid or a salt thereof, pectin and a gas producing material capable of producing a non-toxic gas which when contacted with aqueous acid which forms gastric rafts over a broader pH range and where the gel strength does not depend on the concentration of sugar in the gastrointestinal tract. Also provided is the use of said composition.

### SUMMARY OF THE INVENTION

A gastric raft composition comprising alginic acid or a salt thereof, pectin and a gas producing material capable of producing a non-toxic gas when contacted with aqueous acid is provided.

The use of a gastric raft composition comprising alginic acid or a salt thereof, pectin and a gas producing material capable of producing a nontoxic gas when contacted with aqueous acid in therapy is also provided.

### DETAILED DESCRIPTION OF THE INVENTION

According to the present invention, there is provided a pharmaceutical composition comprising alginic acid or a salt thereof, pectin and a gas producing material capable of producing a non-toxic gas when contacted with aqueous acid; which at low pH forms a floating gastric raft Also provided is the use in therapy of such a composition.

The present invention provides a solution to some of the issues that exist with the gastric raft formulations of the art. The present invention is therefore concerned with providing a composition that simultaneously is capable of forming floating gastric rafts over a broader pH range than previously known for alginate based compositions and where the optimal gel strength is independent of sugar content in the gastrointestinal tract as is currently the situation with pectin based gastric raft compositions.

We have surprising found that a gastric raft composition essentially consisting of alginic acid or a salt thereof, pectin and a gas producing material capable of producing a non-toxic gas when contacted with aqueous acid provides for the formation of floating gastric rafts on ingestion over a broader pH range and of a suitable strength not requiring a specific sugar concentration in the gastrointestinal tract.

The formation of gels from the composition of the present invention relies on the interaction of alginate and pectin. It has been shown that mixtures of alginates and pectins co-operatively associate to form firm resilient gels, in the absence of calcium or high concentrations of sugar, under conditions of low pH. It has also been noted that the presence of calcium ions in the mixture on acidification can be deleterious to the gelling interaction (Thom et al., Prog. Fd. Nutr. Sci., Vol 6 pp97-108, 1982).

The invention therefore provides for a pharmaceutical composition comprising-alginic acid or a salt thereof, pectin and a gas producing material capable of producing a non-toxic gas when contacted with aqueous acid, which in a low pH (i.e. acidic) environment will form floating gastric rafts. The invention is best optimised in the absence calcium ions on acidification as calcium ions can be deleterious to the gelling process. It is thus preferred that the components of the present composition should not liberate calcium ions when exposed to a low pH environment.

We prefer that pectin is high ester pectin containing an ester content of greater than about 50% along the biopolymner chains. More preferably the high ester pectin is methyl ester pectin.

We prefer that alginic acid or the salt thereof is of high guluronate content It is also preferred that alginic acid or the salt thereof is selected from the undissociated acid, sodium alginate or potassium alginate. Preferably, alginic acid is used and most preferably alginic acid of high guluronate content is used.

We prefer that the gas producing material is selected from the carbonate or bicarbonate of an alkali metal or an alkaline earth metal except that of calcium. More preferably the gas producing material is selected from the bicarbonate of an alkali or alkaline earth metal except that of calcium. Even more preferably the gas producing material is selected from sodium bicarbonate or potassium bicarbonate and most preferred is sodium bicarbonate.

We further provide for the addition to the composition of a pharmaceutically active ingredient. This ingredient may be effective in the neutralisation of acid (an antacid).

In a preferred embodiment, alginic acid or a salt thereof and pectin are present in the composition in a ratio of about 1:1.

In a preferred embodiment, the composition comprises alginic acid or a salt thereof present at 50 to 500 mg per unit dose and 2 to 20 wt. % content, high ester pectin present at 50 to 500 mg per unit dose or 2 to 20 wt. % content, bicarbonate of alkali or alkaline earth metal (excluding calcium) present at 50 to 400 mg and 2 to 16 wt. % and a pharmaceutically active ingredient present in an appropriate amount. We prefer that the pharmaceutically active ingredient is an antacid or mixture of antacids.

A preferred composition contains 250 mg alginic acid, 250 mg high methoxy pectin (1:1 as ratio) and 200 mg NaHCO₃.

The composition of the present invention is useful for the treatment of gastrointestinal tract. As such we further provide for the use of said composition in therapy; the use of said composition in the manufacture of a medicament for the treatment of gastrointestinal reflux disease; and a method of treating a patient in need of therapy, comprising administering to said patient a therapeutically effective amount of the composition of the present invention.

The composition may be administered orally in the form of tablets, capsules or powder sachets.

### EXAMPLES

### Example 1

A gastric raft composition comprising:

| | |
|---|---|
| Sodium alginate | 2.5% w/w |
| High methoxy pectin | 2.5% w/w |
| Sodium bicarbonate | 2.0% w/w |
| Water | to 10 ml |

The above composition formed a raft over the pH range 1 to 1.7 in vivo (10 ml of formulation in 100 ml HCl)

### Example 2

A gastric raft composition comprising:

| | |
|---|---|
| Alginic acid | 2.5% w/w |
| High methoxy pectin | 2.5% w/w |
| Sodium bicarbonate | 2.0% w/w |
| Water | to 10 ml |

The above composition formed a raft over the pH range 1 to 2.0 in vivo (10 ml of formulation in 100 ml HCl)

### Example 3

The compositions of examples 1 and 2 were allowed to form rafts in hydrochloric acid at pH 1.6. For each composition, the visco-elastic structure was measured. Visco-elastic structure was characterised using creep rheology measurements taken on the Carri-med CSL 100 rheometer. The gels of both formulations demonstrated increased visco-elastic structure compared to that of the alginate only Gaviscon liquid liquid® formulation.

### Example 4

The composition of examples 1 and 2 were orally administered to human volunteers. Floating gastric rafts were observed by Magnetic Resonance Imaging on the surface of the gastric contents over a test period of 45 minutes. Formation of the rafts was rapid, occurring within 2 minutes of ingestion. All rafts resided on the surface of the gastric contents for the duration of the test period.

1. A pharmaceutical composition comprising:
alginic acid or a salt thereof;
pectin; and
a gas producing material capable of producing a non-toxic gas when contacted with aqueous acid
which is able to form a floating gastric raft when exposed to a low pH.

2. A composition according to claim 1 wherein pectin is selected from high ester pectin containing an ester content of greater than 50% along the biopolymer chains.

3. A composition according to claim 2 wherein the high ester pectin is methyl ester pectin.

4. A composition according to claim 1 wherein the alginic acid or salt thereof is of high guluronate content.

5. A composition according to claim 1 wherein the alginic acid or salt thereof is selected from the undissociated acid, sodium alginate or potassium alginate.

6. A composition according to claim 5 wherein alginic acid is used.

7. A composition according to claim 1 wherein the gas producing material is selected from the carbonate or bicarbonate of an alkali metal or an alkaline earth metal except that of calcium.

8. A composition according to claim 7 wherein the gas producing material is selected from sodium bicarbonate or potassium bicarbonate.

9. A composition according to claim 8 wherein the gas producing material is sodium bicarbonate.

10. A composition according to claim 1 further comprising a pharmaceutically acceptable ingredient which is an antacid.

11. A composition according to claim 1 wherein the ratio of alginic acid or salt thereof to pectin is about 1:1.

12. A composition according to claim 1 wherein alginic acid or the salt thereof is present at 50 mg to 500 mg per unit dose and 2 to 20 wt. % content, high ester pectin is present at 50 to 500 mg per unit dose or 2 to 20 wt. % content, the bicarbonate of an alkali or alkaline earth metal (excluding calcium) is present at 50 to 400 mg and 2 to 16 wt. %.

13. A composition according to claim 12 further comprising an antacid ingredient in an appropriate amount.

14. A composition comprising 250 mg alginic acid, 250 mg high methoxy pectin (1:1 ratio) and 200 mg NaHCO₃.

15. The use of a composition according to claim 1 in therapy.

16. The use of a composition according to claim 1 in the treatment of gastrointestinal reflux disease.

17. The use of a composition according to claim 1 in the manufacture of a medicament for the treatment of gastrointestinal reflux disease.

18. A method of treating a patient in need of therapy, comprising administering to said patient a therapeutically effective amount of a composition as claimed in claim 1.
